(19) United States
(12) Patent Application Publication
(10) Pub. No.: US 2023/0172472 A1
(43) Pub. Date: Jun. 8, 2023

(54) BIOLOGICAL ENERGY SIGNAL ACQUISITION AND CONVERSION DEVICE

(71) Applicants: Chun-Fang Cheng, New Taipei (TW); Ting-Han Hong, Taoyuan (TW); Po-Yuan Kung, Yunlin (TW); Jia-De Ni, Taipei (TW); Hsun-Tsan Shen, New Taipei (TW); Wen-Chun Yeh, Taipei (TW)

(72) Inventors: Chun-Fang Cheng, New Taipei (TW); Ting-Han Hong, Taoyuan (TW); Po-Yuan Kung, Yunlin (TW); Jia-De Ni, Taipei (TW); Hsun-Tsan Shen, New Taipei (TW); Wen-Chun Yeh, Taipei (TW)

(21) Appl. No.: 17/636,648

(22) PCT Filed: Aug. 28, 2019
(86) PCT No.: PCT/CN2019/102998

§ 371 Date: Jun. 16, 2022

(51) Int. Cl. A61B 5/05

(52) U.S. Cl. CPC A61B 5/05

(57) ABSTRACT

A biological energy signal acquisition and conversion device comprises: a signal acquisition module, enhancing a biological energy signal generated by an original biological energy body, and extracting the signal; a signal processing module, receiving the extracted original biological energy signal and filtering and amplifying the signal, and generating an output energy signal; and a signal output unit, being able to enhance an output function in cooperation with a magnetic substance, and output the energy signal to a biological energy carrier. By shortening the distance between the original biological energy body and a signal receiving unit, and using the magnetic substance to excite the original biological energy body, the strength of the received extracted original biological energy signal is enhanced, and the noise proportion is greatly reduced, reducing the technical complexity and costs for back-end signal processing, and improving the effect of a biological energy signal.

### BACKGROUND OF THE INVENTION

### 1. Field of the Invention

The invention is related to a signal acquisition and conversion device, in particular to a biological energy signal acquisition and conversion device.

### 2. Description of the Related Art

This biological energy signal acquisition and conversion device is a device that captures and enhances the energy signal of an original biological energy body and transmits it directly to the biological energy carrier for signal acceptance and conversion, such as the MORA Super+biological energy resonance electronic equipment manufactured by MedTronik, Germany. Back to 2013, Liu Ling-Ling, Cheng Chun-Fang, et al. published a journal paper in European Journal of Integrative Medicine, “Effectiveness of MORA electronic homeopathic copies of remedies for allergic rhinitis: A short-term, randomized, placebo-controlled PILOT study” proposing effectively treating allergic rhinitis by such a device.

However, the strength of the energy signal from the original biological energy body is very weak, many unnecessary environmental noise signals are mixed in the energy signal when the energy signal is captured, and part of the circuit noise is mixed in the energy signal during the enhancement process. Unavoidably, the biological energy transmitted to the biological energy carrier is mixed. It also absorbs uncorrelated noise signals. This proportion of noise signals even exceeds the energy signal of the original biological energy body to be transmitted. It greatly reduces the effect of characteristic signals of the biological energy to be received by the biological energy carrier.

In order to solve the problem of uncorrelated noise signals, the signal capturing method of the existing biological energy signal capturing and converting device must be equipped with a complex signal processing circuit to process various biological energy characteristic signals. Some are even connected to a computer and use software for signal-assisted processing, but this causes the complexity of the technical architecture and greatly increases the cost. It is critical to improve the conventional biological energy signal acquisition and conversion device.

### SUMMARY OF THE INVENTION

In view of the poor design of the front-end signal acquisition processing in the conventional biological energy signal acquisition and conversion device, the complexity of the back-end signal processing method and the price both increase. The present invention provides a biological energy signal acquisition and conversion device. It includes a signal acquisition module, a signal processing module and a signal output unit. The signal acquisition module is used to enhance and intensify the biological energy signal generated by an original biological energy body, and to capture the enhanced and stimulated biological energy signal. The former biological energy bodies include energy bodies and biological energy carriers, which are composed of all or parts of tissues and derivatives of the biological energy bodies. The derivatives include biological fluids, secretions, excreta, their normal or pathological biological derivatives, and epidermal keratin of normal, or infected-related pathological substances. The normal or pathological biological derivatives might be blood, sputum, nasal mucus, urine, pus, etc., and the epidermal keratin may be hair, nails, dander, etc. The biological energy signal can be extracted for processing, such as duplicating, summing, filtering, or reducing. The signal processing module is electrically connected to the signal acquisition module to receive the original biological energy signal and process filtering and amplifying on the original biological energy signal to generate an output energy signal. The signal output unit is electrically connected to the signal processing module to receive the output energy signal and transmit the output energy signal to a biological energy carrier, and proposes a better conversion method to improve the carrier's efficiency of carrying the converted biological energy signal.

The signal acquisition module can greatly reduce noise and improve the signal acquisition method through a better design. First, the signal acquisition module can receive the enhanced and stimulated biological energy signal. Secondary it can significantly reduce the noise ratio of the original biological energy signal captured by the signal acquisition module. Finally the signal processing complexity of the back-end signal processing module can be reduced, and achieve the purpose of reducing the technical cost and simplifying operational difficulty of the overall device.

The signal output unit proposes a better designed conversion structure to shorten the distance between the biological energy carrier and the signal output unit, so as to enhance the receiving efficiency of the biological energy carrier, and maximize the efficiency of converting the biological energy signal to the biological energy carrier.

### BRIEF DESCRIPTION OF THE DRAWINGS

FIG. 1 is a three-dimensional schematic diagram of the biological energy signal acquisition and conversion device of the present invention.

FIG. 2 is a block diagram of the biological energy signal acquisition and conversion device of the present invention.

FIG. 3 is a schematic cross-sectional view of the carrying box of the biological energy signal acquisition and conversion device of the present invention.

FIG. 4 is a three-dimensional schematic diagram of the carrying box of the biological energy signal acquisition and conversion device of the present invention.

FIG. 5 is a three-dimensional schematic diagram of the signal output unit of the biological energy signal acquisition and conversion device of the present invention.

FIG. 6 is a three-dimensional schematic diagram of the application of the biological energy signal acquisition and conversion device of the present invention.

FIG. 7 is a block diagram of a second preferred embodiment of the biological energy signal acquisition and conversion device of the present invention.

### DETAILED DESCRIPTION OF THE INVENTION

The following describes the present invention in detail with reference to the accompanying drawings and specific embodiments, but it is not intended to limit the present invention.

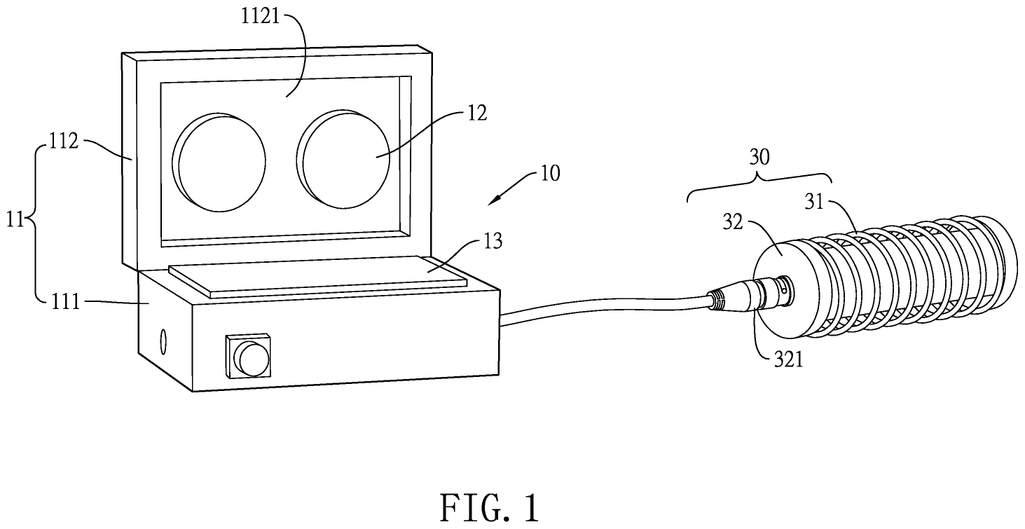

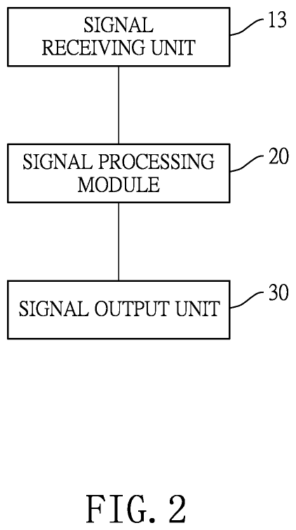

Please refer to FIG. 1 and FIG. 2. In view of the poor design of the conventional front-end signal capture processing of the biological energy signal acquisition and conversion device, the back-end signal processing method is complicated and the cost will increase. The present invention provides a biological energy signal acquisition and conversion device. The biological energy signal acquisition and conversion device includes a signal acquisition module 10, a signal processing module 20, and a signal output unit 30. The signal acquisition module 10 is used to capture a biological energy signal generated by an original biological energy body, and the signal acquisition module 10 first performs enhancement and activation of the original biological energy body, and then receives an enhanced and activated original biological energy signal. The signal processing module 20 is electrically connected to the signal acquisition module 10 to receive the original biological energy signal and processes filtering and amplifying on the original biological energy signal to generate an output energy signal. The signal output unit 30 is electrically connected to the signal processing module 20 to receive the output energy signal and transmit the output energy signal to a biological energy carrier.

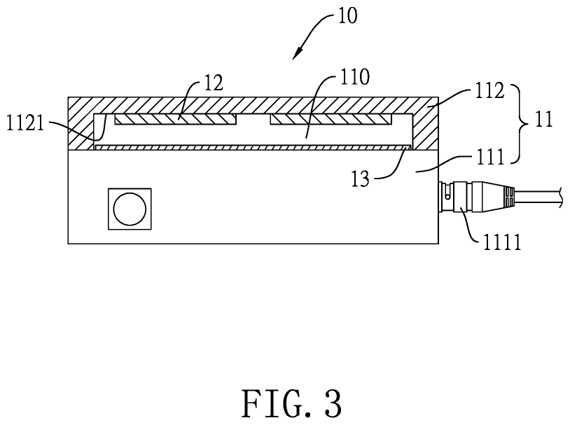

Please also refer to FIG. 1 and FIG. 3 together. In a first preferred embodiment of the present invention, the signal acquisition module 10 includes a carrying box 11, a first magnetic unit 12 and a signal receiving unit 13. The carrying box 11 includes a base 111 and an upper cover 112. One side of the upper cover 112 is pivotally connected to the base 111 and can be pivotally closed with the base 111. Wherein, the first magnetic unit 12 is arranged on an inner surface of the upper cover 112, and the signal receiving unit 13 is arranged on an upper surface of the base 111 for carrying an original biological energy body and receiving the original biological energy signal produced by the original biological energy body. When the upper cover 112 and the base 111 are pivotally closed, there is an accommodating space 110 between the first magnetic unit 12 and the signal receiving unit 13. The signal processing module 20 is disposed in the internal space of the base 111 and is electrically connected to the signal receiving unit 13. The signal processing module 20 receives the original biological energy signal from the signal receiving unit 13, and performs filtering and amplification on the original biological energy signal to generate the output energy signal. The signal output unit 30 is electrically connected to the signal processing module 20 to receive the output energy signal and transmit the output energy signal to the biological energy carrier.

When the upper cover 112 and the base 111 are closed, the original biological energy body is located in the accommodating space 110 and between the signal receiving unit 13 and the first magnetic unit 12. The first magnetic unit 12 can approach the original biological energy body at a similar short distance, use the physical characteristics of the magnetic field distance of the magnetic substance, and excite the original biological energy body with the largest magnetic field formed by the short distance of the first magnetic unit 12 to generate the largest biological energy signal. The signal receiving unit 13 directly receives the enhanced original biological energy signal, so that the ratio of the original biological energy signal in the whole signal received by the signal receiving unit 13 is increased. Therefore, the ratio of noise from the surrounding environment is greatly reduced. In short, by shortening the distance between the signal receiving unit 13 and the original biological energy body, and further using the first magnetic unit 12 to excite the original biological energy body, the original biological energy signal received by the signal receiving unit 13 is strengthened and the noise proportion is greatly reduced.

In addition, the original biological energy body is placed in the carrying box 11 and protected by the upper cover 112, it won't easily encounter with external force . In this way, through the improved structure of the carrying box 11 and the activation of the original biological energy by the first magnetic unit 12, the signal quality received by the front-end signal receiving unit 13 is greatly improved, thereby further reducing the technical difficulty and cost required by the back-end signal processing. The original biological energy body includes all or parts of the tissues of the organism, or derivatives of the organism, such as biological fluids, secretions, excreta, and includes its normal or pathological biological derivatives, such as blood, sputum, nasal mucus, urine, pus, etc.; normal, or infected-related pathological substances of epidermal keratin, such as hair, nails, dander, etc. which constitute the energy body. The original biological energy body may also be a biological energy carrier. For example, the biological energy carrier that has received the biological energy signal in the present invention can be put on the signal receiving unit 13 to extract the energy signal again.

In a first preferred embodiment of the present invention, the signal output unit 30 includes a coil 31 that is electrically connected to the signal processing module 20 to receive the output energy signal and send the output energy signal. Preferably, the coil 31 is electrically connected to the signal processing module 20 provided in the inner space of the base 111 through a wire.

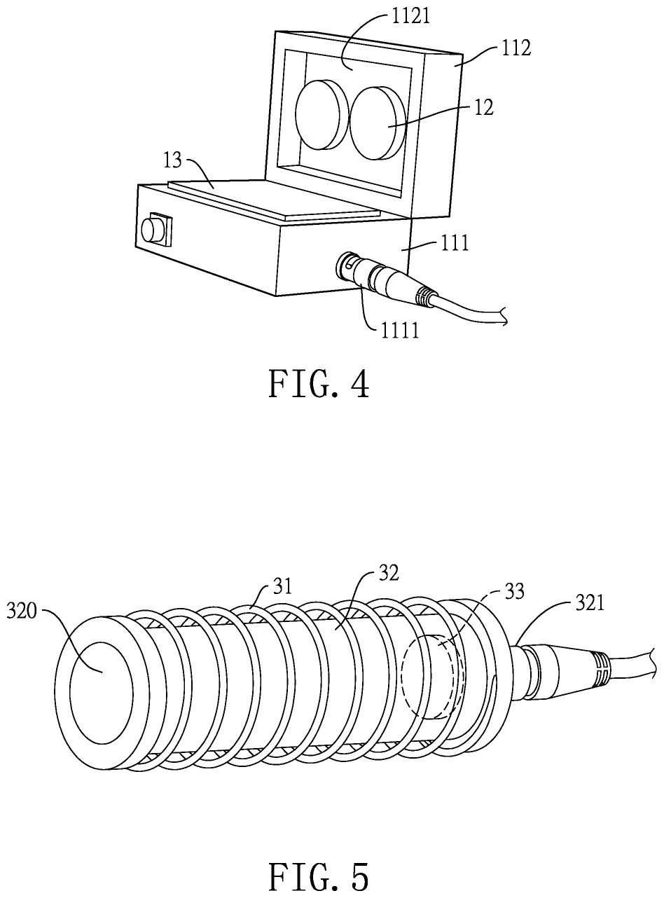

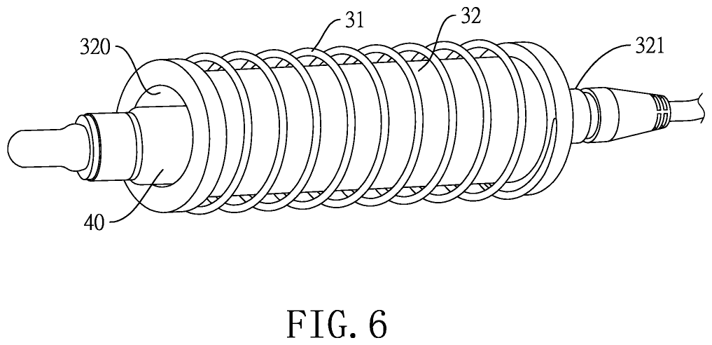

Please refer to FIG. 1, FIG. 4 and FIG. 5 together. Preferably, an output terminal 1111 is provided on a side wall of the base 111 of the carrying box 11, and the output terminal 1111 is electrically connected to the signal processing module 20 to output the output energy signal. The signal output unit 30 still includes a tube body 32, preferably a hollow tube body. The tube body 32 has a carrier accommodating space 320, and one of the opposite ends of the tube body 32 is formed with an opening. The other end is provided with an input terminal 321.The input terminal 321 is electrically connected to the output terminal 1111 of the base 111 through the wire, and the opening of the tube body 32 communicates with the carrier accommodating space 320 of the tube body 32. The coil 31 is wound around the tube body 32 and is electrically connected to the input terminal 321 to receive the output energy signal output by the signal processing module 20 through the wire and the output terminal 1111 of the base 111. The carrier accommodating space 320 is for placing a biological energy carrier, and the biological energy carrier receives the output energy signal of the coil 31. Referring to FIG. 6, the biological energy carrier is preferably a bottled liquid 40 such as wine, vinegar, water or a solid material such as quartz, crystal, crystalline structure, salt, sugar or magnetic substance.

In another embodiment, the carrier accommodating space 320 of the tube body 32 may not contain a carrier. At this time, the coil 31 is used to directly send the output energy signal through the opening of the tube body 32 to an external biological energy carrier within a certain distance. The biological energy carrier includes an animal or a human body.

Preferably, the signal receiving unit 13 is a metal plate made of gold, silver, or copper.

In more detail, the inner surface of the upper cover 112 is concavely formed with an embedding groove 1121, and the first magnetic unit 12 is disposed in the embedding groove 1121, and is preferably fixed by bolts. The bolts are pasted or fitted into the embedding groove 1121. Wherein, the signal receiving unit 13 is disposed corresponding to the embedding groove 1121, and the thickness of the signal receiving unit 13 plus the thickness of the first magnetic unit 12 is less than the depth of the embedding groove 1121. Therefore, when the upper cover 112 and the base 111 are closed, the signal receiving unit 13 will be kept in the embedding groove 1121, but never contact the first magnetic unit 12, and will form a short distance. The accommodating space 110 with a fixed height is formed between the signal receiving unit 13 and the first magnetic unit 12 for accommodating the biological energy body.

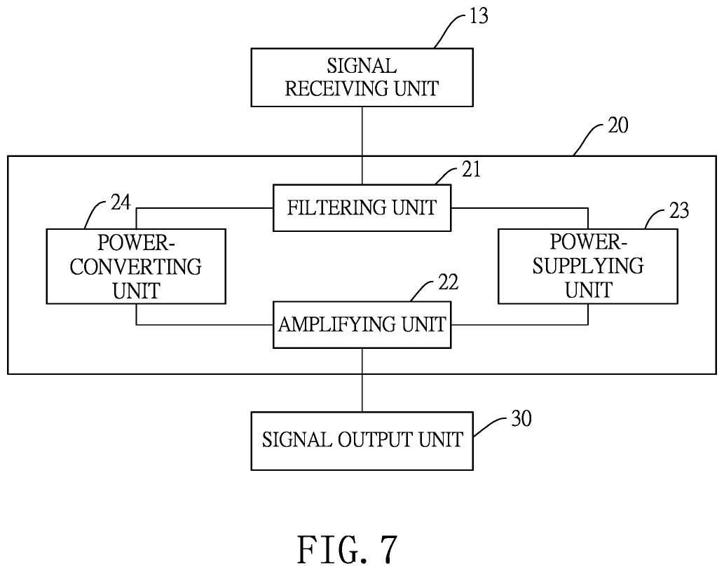

Referring to FIG. 7, in a second preferred embodiment of the present invention, the signal processing module 20 includes a filtering unit 21 and an amplifying unit 22. The filtering unit 21 is electrically connected to the signal receiving unit 13 to receive the original biological energy signal. The related noise that does not belong to the range of the biological energy signal is greatly attenuated and removed by the filtering unit 21, such as filtering out low-frequency 50/60 Hz AC power waves, a high-frequency mobile phone or radio wave environmental noise, etc., to produce an output filtered signal. The amplifying unit 22 is electrically connected to the filtering unit 21 and receives the output filtered signal and performs an amplifying process to generate the output energy signal. The output terminal 1111 is electrically connected to the amplifying unit 22 for outputting the output energy signal.

Preferably, the signal processing module 20 includes a power-supplying unit 23 disposed in the inner space of the base 111, and the power-supplying unit 23 is used to provide the power required by the filtering unit 21 and the amplifying unit 22. The power-supplying unit 23 is, for example, a battery.

In another embodiment, the signal processing module 20 includes a power-converting unit 24, electrically connected to the filtering unit 21 and the amplifying unit 22, and electrically connected to an external power source to convert the external power source and provide power to the filtering unit 21 and the amplifying unit 22.

Please refer to FIG. 5 again. In a third preferred embodiment of the present invention, the signal output unit 30 further includes a second magnetic unit 33, which can be disposed in the accommodating space 320 of the tube body 32. It is placed in the accommodating space 320 to enhance the effectiveness of the coil 31. Depending on type or structure of the biological energy carrier, the second magnetic unit 33 can also use the physical characteristics of the magnetic field distance of the magnetic substance to be placed around the coil 31 or other positions inside the tube body 32 or around the outer side of the tube body 32 and close to coils, and other locations around or inside the biological energy carrier 40.

The above are only preferred embodiments of the present invention, and do not limit the present invention in any form. Although the present invention has been disclosed as above in preferred embodiments, it is not intended to limit the present invention.

The present invention strengthens the biological energy signal generated by the original biological energy body through a signal acquisition module and performs the signal extraction. The signal processing module receives and extracts the original biological energy signal and processes filtering and amplifying on the signal to generate an output energy signal. The output energy signal is generated by the signal output unit. With magnetic material, it can strengthen the output function to output energy signal to the biological energy carrier. By shortening the distance between the original biological energy body and the signal receiving unit, and using the magnetic material to enhance and intensify the original biological energy body, the original biological energy can be received and extracted. The signal intensity is enhanced and the noise proportion is greatly reduced, which reduces the technical complexity and the cost of the back-end signal processing and improves the efficiency of converting the biological energy signal to the biological energy carrier.

Even though numerous characteristics and advantages of the present invention have been set forth in the foregoing description, with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A biological energy signal acquisition and conversion device, characterized in that the device comprises:
a signal acquisition module, comprising a first magnetic unit, for enhancing a biological energy signal, exciting an original biological energy body, and extracting an original biological energy signal;
a signal processing module, comprising a filtering unit and an amplifying unit, electrically connected to the signal acquisition module to receive the original biological energy signal and process filtering and amplifying on the original biological energy signal to generate an output energy signal; and
a signal output unit, comprising a coil, which is electrically connected to the signal processing module to receive the output energy signal and transmit the output energy signal to a biological energy carrier,
wherein the original biological energy body is a normal or pathological organism, an energy body constituted by biological derivatives, or the biological energy carrier.

2. The biological energy signal acquisition and conversion device of claim 1, wherein the signal acquisition module comprises:
a carrying box; and
a signal receiving unit arranged in the carrying box and receiving the original biological energy signal;
wherein the first magnetic unit is arranged in the carrying box and corresponding to the signal receiving unit, and an accommodating space is formed between the first magnetic unit and the signal receiving unit; wherein,
the signal processing module is arranged in the carrying box and electrically connected to the signal receiving unit.

3. The biological energy signal acquisition and conversion device of claim 2, wherein the carrying box body is provided with an output terminal, and the output terminal is electrically connected to the signal processing module, and the signal output unit further includes:
a tube body, having a carrier accommodating space, one of two opposite ends of the tube body formed with an opening, and the other end of the tube body provided with an input terminal which is electrically connected to the output terminal of the processing module through a wire, the opening of the tube body is connected to the carrier accommodating space of the tube body; the coil is wound around the tube body and is electrically connected to the input terminal.

4. The biological energy signal acquisition and conversion device of claim 2, wherein
the filtering unit is electrically connected to the signal receiving unit to receive the original biological energy signal, and performs a filtering process on the original biological energy signal to generate a filtered signal; and
the amplifying unit is electrically connected to the filtering unit to receive the filtered signal, and the amplifying unit performs an amplifying process on the filtered signal to generate the output energy signal.

5. The biological energy signal acquisition and conversion device of claim 3, wherein the signal output unit further comprises:
a second magnetic unit, arranged in the carrier accommodating space of the tube body, in the tube body or around the outer side of the tube body and close to the coil.

6. The biological energy signal acquisition and conversion device of claim 4, wherein the signal processing module further comprises:
a power-supplying unit electrically connected to the filtering unit and the amplifying unit to generate power to supply the filtering unit and the amplifying unit.

7. The biological energy signal acquisition and conversion device of claim 1, wherein:
the biological derivatives are body fluids, secretions, excreta, and epidermal keratin of normal or diseased organisms.

8. The biological energy signal acquisition and conversion device of claim 1, wherein:
the biological energy carrier is bottled liquid, a solid material with a crystalline structure or a magnetic substance.
